(19) United States
(12) Patent Application Publication
(10) Pub. No.: US 2015/0148486 A1
(43) Pub. Date: May 28, 2015

(54) METHOD OF IMMOBILIZING BIOLOGICALLY ACTIVE SUBSTANCE

(71) Applicant: SUMITOMO BAKELITE COMPANY, LTD., Tokyo (JP)

(72) Inventors: Sohei FUNAOKA, Tokyo (JP); Midori ABE, Tokyo (JP)

(73) Assignee: SUMITOMO BAKELITE COMPANY, LTD., Tokyo (JP)

(21) Appl. No.: 14/610,681

(22) Filed: Jan. 30, 2015

Related U.S. Application Data
(62) Division of application No. 12/921,664, filed on Nov. 5, 2010.

(51) Int. Cl. G01N 33/531

(52) U.S. Cl. CPC G01N 33/531
USPC 525/54.1

(57) ABSTRACT

An object of the present invention is to provide a method of immobilizing the biologically active substance which has an excellent capability of immobilizing a target biologically active substance, and exhibits low nonspecific adsorption of the biologically active substance to provide a high S/N ratio, without using a functional group for fixing the biologically active substance and without having a process of inactivating the functional group for fixing the biologically active substance after immobilizing the biologically active substance. The above object is achieved by a method of immobilizing a biologically active substance comprising the step of: bringing a solution into contact with a compound-side surface of an immobilizing substrate to immobilize the biologically active substance on a surface of the immobilizing substrate, the solution being prepared by dissolving the biologically active substance in a phosphate buffer having a phosphate concentration of 0.1 M or more, and the immobilizing substrate comprising a substrate and a compound containing a hydrophilic group inhibiting nonspecific adsorption on a surface of the substrate.

### TECHNICAL FIELD

The present invention relates to a method of immobilizing a biologically active substance.

### BACKGROUND ART

Traditionally, attempts to evaluate gene activity and to decode biological process including disease process, and biological process of drug effect have focused on genomics. However, proteomics can provide more detailed information on biological functions of a cell. The proteomics includes qualitative and quantitative measurement of gene activity by detecting and quantitating expression at a protein level rather than a gene level, and also includes research of matters which are not encoded by a gene, such as post-translational modification of proteins and interaction between proteins.

Today, an enormous amount of genome information genome is available and faster and higher efficiency (higher throughput) of proteomics research is required. As a molecule array to achieve the above, a DNA chip has been put into practical use. On the other hand, regarding detection of a protein which is the most complicated and has high diversity in biological function, a protein chip has been proposed and the research thereof has recently been advancing. The protein chip is a generic term for a chip (a minute substrate or a particle) on the surface of which a protein or a molecule for capturing the protein is immobilized.

However, the present protein chips have generally been developed as an extension of development of the DNA chip, therefore, studies have been carried out into immobilization of the protein or the molecule for capturing the protein on a glass substrate or a particle as a chip surface (for example, see Patent Literature 1).

In signal detection of the protein chip, nonspecific adsorption of a target substance to be detected onto the substrate can be exemplified as a cause of decreasing a signal/noise ratio (for example, see Non Patent Literature 1).

As a method of immobilization, two types of methods are performed. One of the methods is a method of immobilizing a protein by physical adsorption. In this method, the surface of the substrate easily absorbs the protein, therefore, coating of an adsorption inhibitor is performed after immobilizing the protein to prevent nonspecific adsorption of an antigen and/or a second antibody. However, the ability of preventing nonspecific adsorption is insufficient. In addition, there is a problem that the coating of the adsorption inhibitor is performed after immobilizing a first antibody so that the immobilized proteins are coated with the adsorption inhibitor, thereby, the immobilized proteins cannot react with the second antibody. Thus, there has been a desire for a substrate for bioassay, which does not require the coating of the adsorption inhibitor after immobilizing the first antibody, and has less amount of nonspecific adsorption of the biologically active substance.

The other method is a method of binding a protein to the surface using a functional group. The functional group which reacts with the protein is introduced into a component for forming matrix in which the protein is less likely to be absorbed, and the protein is immobilized via the functional group (for example, see Patent Literature 2). However, even in the method of immobilizing the biologically active substance as disclosed in Patent Literature 2, capability of immobilizing a target biologically active substance may be poor, and an S/N ratio may be insufficient due to high nonspecific adsorption. Also, in the case of using the functional group which reacts with the protein, there is a problem that it takes time to proceed to the next step because of the needs of the process of immobilizing the protein and the process of inactivating the functional group which reacts with the protein after immobilizing. Therefore, a method not using the functional group which reacts with the protein has been also desired.

### CITATION LIST

[Patent Literature 1] Japanese Patent Application Laid-Open (JP-A) No. 2001-116750
[Patent Literature 2] Published Japanese translation of a PCT application No. 2004-531390
[Non Patent Literature 1]“Practical manual of DNA microarray”, Yoshihide Hayashizaki, Yasushi Okazaki, YODOSHA Co., Ltd., 2000, p. 57.

### SUMMARY OF INVENTION

### Technical Problem

A first object of the present invention is to provide a method of immobilizing a biologically active substance which has an excellent capability of immobilizing a target biologically active substance, exhibits low nonspecific adsorption of the biologically active substance and provides a high S/N ratio.

A second object of the present invention is to provide a method of immobilizing the biologically active substance, in which a functional group for fixing the biologically active substance is not used so that a process of inactivating the functional group for fixing the biologically active substance after immobilizing the biologically active substance is not required, in addition to having the features mentioned in the first object.

### Solution to Problem

The present invention can be attained by the following (1) to (19).

(1) A method of immobilizing a biologically active substance comprising the step of: bringing a solution into contact with a compound-side surface of an immobilizing substrate to immobilize the biologically active substance on a surface of the immobilizing substrate, the solution being prepared by dissolving the biologically active substance in a phosphate buffer having a phosphate concentration of 0.1 M or more, and the immobilizing substrate comprising a substrate and a compound containing a hydrophilic group inhibiting nonspecific adsorption on a surface of the substrate.

(2) The method of immobilizing the biologically active substance according to (1), wherein the hydrophilic group inhibiting nonspecific adsorption is an alkylene glycol residue and/or a phosphorylcholine group.

(3) The method of immobilizing the biologically active substance according to (1) or (2), wherein the compound is a polymer compound containing a repeating unit (A) having an alkylene glycol residue or a phosphorylcholine group.

(4) The method of immobilizing the biologically active substance according to (3), wherein the repeating unit (A) having the alkylene glycol residue or the phosphorylcholine group is derived from an ethylenically unsaturated polymerizable monomer represented by the following formula [1]:

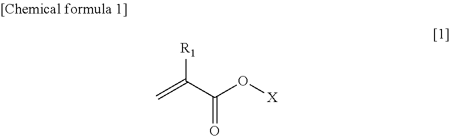

wherein R₁ represents a hydrogen atom or a methyl group; and X represents a group having an alkylene glycol residue or a phosphorylcholine group.

(5) The method of immobilizing the biologically active substance according to (4), wherein the repeating unit (A) having the alkylene glycol residue is derived from an ethylenically unsaturated polymerizable monomer having an alkylene glycol residue represented by the following formula [1′]:

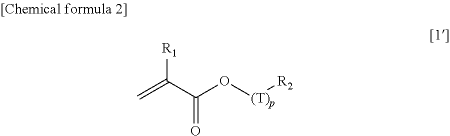

wherein R₁ represents a hydrogen atom or a methyl group; R₂ represents a hydrogen atom or an alkyl group having 1 to 20 carbon atoms; T represents an alkylene glycol residue having 1 to 10 carbon atoms; p represents an integer from 1 to 100; and provided that when p is an integer of 2 or more and 100 or less, the repeated Ts may be the same or different from each other.

(6) The method of immobilizing the biologically active substance according to (5), wherein the ethylenically unsaturated polymerizable monomer having the alkylene glycol residue is methoxy polyethylene glycol(meth)acrylate.

(7) The method of immobilizing the biologically active substance according to (6), wherein an average chain of ethylene glycol in the methoxy polyethylene glycol(meth)acrylate is in the range from 3 to 100.

(8) The method of immobilizing the biologically active substance according to any of (3) to (7), wherein the polymer compound contains a repeating unit (B) having a crosslinkable functional group.

(9) The method of immobilizing the biologically active substance according to (8), wherein the crosslinkable functional group of the repeating unit (B) having the crosslinkable group is at least one functional group selected from alkoxysilyl, epoxy, and (meth)acrylic groups.

(10) The method of immobilizing the biologically active substance according to (8) or (9), wherein the repeating unit (B) having the crosslinkable functional group is an ethylenically unsaturated polymerizable monomer having alkoxysilyl represented by the following formula [2]:

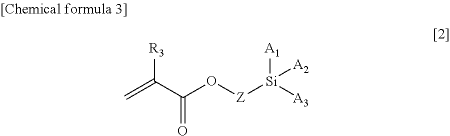

wherein R₃ represents a hydrogen atom or a methyl group; Z represents an alkyl group having 1 to 20 carbon atoms; and among A₁, A₂, and A₃, at least one is an alkoxy group capable of being hydrolyzed, and the others represent an alkyl group.

(11) The method of immobilizing the biologically active substance according to any of (1) to (10), wherein the compound contains a functional group for fixing the biologically active substance.

(12) The method of immobilizing the biologically active substance according to (11), wherein the compound containing the functional group for fixing the biologically active substance is a polymer compound containing a repeating unit (C) having the functional group for fixing the biologically active substance derived from an ethylenically unsaturated polymerizable monomer having an active ester group represented by the following formula [3]:

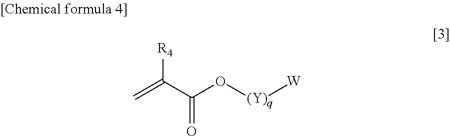

wherein R₄ represents a hydrogen atom or a methyl group; Y represents an alkylene group or alkylene glycol residue having 1 to 10 carbon atoms; W represents an active ester group; q represents an integer from 1 to 20; and provided that when q is 2 or more, the repeated Ys may be the same or different from each other.

(13) The method of immobilizing the biologically active substance according to (12), wherein the functional group for fixing the biologically active substance is a p-nitrophenyl ester group.

(14) The method of immobilizing the biologically active substance according to any of (1) to (13), wherein the phosphate concentration of the phosphate buffer is 5M or less.

(15) The method of immobilizing the biologically active substance according to any of (1) to (14), wherein the biologically active substance is a nucleic acid, an aptamer, a protein, an antibody, an antigen, a lectine, a glycoprotein, or a sugar chain.

(16) The method of immobilizing the biologically active substance according to any of (1) to (15), wherein the substrate is made of plastic.

(17) The method of immobilizing the biologically active substance according to (16), wherein the plastic is saturated cyclic polyolefin, polyolefin, or polystyrene.

(18) The method of immobilizing the biologically active substance according to any of (1) to (15), wherein the substrate is made of glass.

(19) The method of immobilizing the biologically active substance according to any of (1) to (18), wherein the substrate is in the form of a slide, a 96-well plate, a 384-well plate, a 1536-well plate, a microchannel, a bead, a tube, or a container.

### Advantageous Effects of Invention

According to the present invention, it is possible to provide a method of immobilizing the biologically active substance which has an excellent capability of immobilizing a target biologically active substance, and exhibits low nonspecific adsorption of the biologically active substance to provide a high S/N ratio, without using a functional group for fixing the biologically active substance and without having a process of inactivating the functional group for fixing the biologically active substance after immobilizing the biologically active substance.

In the present invention, the functional group for fixing the biologically active substance may be used. Even in this case, it is possible to provide a method of immobilizing the biologically active substance which has more excellent capability of immobilizing a target biologically active substance, and exhibits lower nonspecific adsorption of the biologically active substance to provide a higher S/N ratio than before.

### DESCRIPTION OF EMBODIMENTS

A method of immobilizing a biologically active substance of the present invention is a method of immobilizing a biologically active substance comprising the step of: bringing a solution into contact with a compound-side surface of an immobilizing substrate to immobilize the biologically active substance on a surface of the immobilizing substrate, the solution being prepared by dissolving the biologically active substance in a phosphate buffer having a phosphate concentration of 0.1 M or more, and the immobilizing substrate comprising a substrate and a compound containing a hydrophilic group inhibiting nonspecific adsorption on a surface of the substrate.

According to the method of immobilizing the biologically active substance of the present invention, the solution prepared by dissolving the biologically active substance in the phosphate buffer having the phosphate concentration of 0.1 M or more is brought into contact with the compound-side surface of the immobilizing substrate comprising the compound containing the hydrophilic group inhibiting nonspecific adsorption, thereby capability of immobilizing the target biologically active substance can be excellent, and low nonspecific adsorption of the biologically active substance can be exhibited, and the high S/N ratio can be provided, without using the functional group for fixing the biologically active substance. In the present invention, it is presumed that by using the phosphate buffer having significantly higher phosphate concentration of 0.1 M or more than that of a phosphate buffered saline which is conventionally used, the hydrophilic group inhibiting nonspecific adsorption such as a phosphorylcholine group and an alkylene glycol residue swells or gelates, and then the biologically active substance is prone to be physically incorporated into the immobilizing substrate. Thereby, capability of immobilizing the biologically active substance increases without using the functional group for fixing the biologically active substance. In the case of using no functional group for fixing the biologically active substance, there is an advantage that the process of inactivating the functional group for fixing the biologically active substance after immobilizing the biologically active substance is not required.

Examples of a material of the substrate used in the present invention include glass, plastic, metal, etc. From the viewpoint of easiness of surface treatment and mass production, plastic is preferable, and a thermoplastic resin is more preferable.

As the thermoplastic resin, one having less amount of spontaneous fluorescence emission is preferable. For example, it is preferable to use linear polyolefin such as polyethylene or polypropylene, saturated cyclic polyolefin, styrene, or a fluorine-containing resin, and it is more preferable to use the saturated cyclic polyolefin particularly excellent in heat resistance, chemical resistance, low fluorescence, and formability. The term “saturated cyclic polyolefin” as used herein refers to a saturated polymer in which a homopolymer having a cyclic olefin structure or a copolymer of cyclic olefin and α-olefin is hydrogenated.

In order to increase adhesion between the surface of the substrate and the compound, with which the surface is covered or attached, it is preferable to activate the surface of the substrate. As means of the activation, a method of plasma treatment under conditions of oxygen atmosphere, argon atmosphere, nitrogen atmosphere, or air atmosphere, and a method of treatment using an excimer laser such as ArF or KrF can be exemplified. The method of plasma treatment under the condition of oxygen atmosphere is preferable.

The compound, with which the surface of the substrate is covered or attached, contains the hydrophilic group inhibiting nonspecific adsorption. The hydrophilic group inhibiting nonspecific adsorption is not particularly limited as long as it has property of inhibiting nonspecific adsorption of the biologically active substance, and the alkylene glycol residue and/or the phosphorylcholine group can be suitably used. The term “alkylene glycol residue” as used herein refers to an alkyleneoxy group (—R—O—; “R” as used herein refers to an alkylene group), which remains after the condensation reaction of a hydroxyl group at one of chain ends or both chain ends of alkylene glycol (HO—R—OH; “R” as used herein refers to an alkylene group) and other compounds. For example, the alkylene glycol residue in the case of methyleneglycol (HO—CH₂—OH) is a methyleneoxy group (—CH₂—O—), and the alkylene glycol residue in the case of ethylene glycol (HO—CH₂CH₂—OH) is an ethyleneoxy group (—CH₂CH₂—O—).

As the compound containing the alkylene glycol residue and/or the phosphorylcholine group, various kinds of compounds can be exemplified, and a polymer compound containing a repeating unit (A) having the alkylene glycol residue or the phosphorylcholine group is preferable. The above-mentioned polymer compound is a polymer having property of inhibiting nonspecific adsorption of the biologically active substance, and the alkylene glycol residue and/or the phosphorylcholine group play a role of inhibiting nonspecific adsorption of the biologically active substance. In the case of such a polymer compound, function of inhibiting nonspecific adsorption is more excellent, therefore, background detection easily decreases, compared with the case of using a component for forming matrix in a low molecular in which the protein is less likely to be absorbed as disclosed in Patent Literature 2. In particular, the polymer compound (copolymer) containing the repeating unit (A) having the alkylene glycol residue or the phosphorylcholine group and a repeating unit (B) having a crosslinkable functional group is preferable. Furthermore, the crosslinkable functional group plays a role of imparting insolubility to a solvent by making the polymer compounds crosslink after the surface of the substrate is covered or attached with the crosslinkable functional group, and thus, decline in signal due to washing of the substrate can be prevented.

The repeating unit (A) having the alkylene glycol residue or the phosphorylcholine group is preferably one derived from an ethylenically unsaturated polymerizable monomer represented by the following formula [1]:

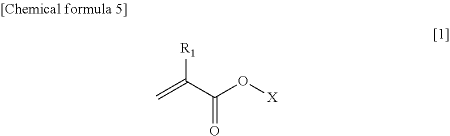

wherein R₁ represents a hydrogen atom or a methyl group; and X represents a group having an alkylene glycol residue or a phosphorylcholine group.

Among them, the structure of the ethylenically unsaturated polymerizable monomer having the alkylene glycol residue used in the present invention is not particularly limited, and one derived from an ethylenically unsaturated polymerizable monomer having an alkylene glycol residue represented by the following formula [1′] is preferable:

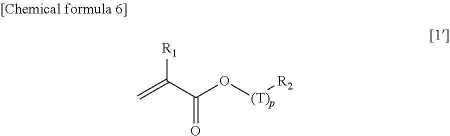

wherein R₁ represents a hydrogen atom or a methyl group; R₂ represents a hydrogen atom or an alkyl group having 1 to 20 carbon atoms; T represents an alkylene glycol residue having 1 to 10 carbon atoms; p represents an integer from 1 to 100; and provided that when p is an integer of 2 or more and 100 or less, the repeated Ts may be the same or different from each other.

The alkylene glycol residue T in the above formula has 1 to 10 carbon atoms, preferably 1 to 6 carbon atoms, more preferably 2 to 4 carbon atoms, still more preferably 2 to 3 carbon atoms, and most preferably 2 carbon atoms. The repeating number “p” of the alkylene glycol residue Ts is an integer from 1 to 100, preferably from 2 to 100, more preferably from 2 to 95, and most preferably from 20 to 90. In the case of using a mixture of the compound having various kinds of “p”, the number “p” of the polymer is specified as the average value of the above. When the repeating number is 2 or more, the carbon atoms of the repeated alkylene glycol residue T may be the same or different from each other.

Examples of the ethylenically unsaturated polymerizable monomer having the alkylene glycol residue include methoxy polyethylene glycol(meth)acrylate; (meth)acrylates of the ester, wherein the hydroxyl group is mono-substituted, such as 2-hydroxyethyl(meth)acrylate, 2-hydroxypropyl(meth)acrylate, and 2-hydroxybutyl(meth)acrylate; glycerol mono(meth)acrylate; (meth)acrylate having polypropylene glycol as its side chain; 2-methoxyethyl(meth)acrylate; 2-ethoxyethyl(meth)acrylate; methoxydiethylene glycol(meth)acrylate; ethoxydiethylene glycol(meth)acrylate; and ethoxy polyethylene glycol(meth)acrylate. From the viewpoint of availability, methoxy polyethylene glycol(meth)acrylate is preferable. Among the above, methoxy polyethylene glycol(meth)acrylate wherein an average chain of ethylene glycol is from 3 to 100 is particularly preferable.

On the other hand, examples of the monomer having the phosphorylcholine group in the formula [1] include 2-(meth)acryloyloxyethyl phosphorylcholine, 2-(meth)acryloyloxyethoxyethyl phosphorylcholine, 6-(meth)acryloyloxyhexyl phosphorylcholine, 10-(meth)acryloyloxyethoxynonyl phosphorylcholine, and 2-(meth)acryloyloxypropyl phosphorylcholine. From the viewpoint of availability, 2-methacryloyloxyethyl phosphorylcholine is preferable.

In the case that the polymer compound contains the repeating unit (A) having the phosphorylcholine group, since the phosphorylcholine group is ampholyte ion, the adhesion with the substrate is excellent if the polymer compound contains no repeating unit having the crosslinkable functional group that will be described hereinafter.

A composition ratio of the repeating unit (A) having the alkylene glycol residue in the polymer compound is preferably 25 to 99 mol %, more preferably 60 to 98 mol %, and still more preferably 70 to 97 mol %.

A composition ratio of the repeating unit (A) having the phosphorylcholine group in the polymer compound is preferably 2 to 50 mol %, more preferably 5 to 45 mol %, and still more preferably 10 to 40 mol %.

The repeating unit (B) having the crosslinkable functional group is not particularly limited as long as it is one which is introduced so as not to develop a reaction of the crosslinkable functional group during synthesis of the polymer. A polymer compound may be produced using an ethylenically unsaturated polymerizable monomer having the crosslinkable functional group, or the crosslinkable functional group may be introduced into a polymer compound using a reactive functional group appropriately, for example, using a combination of a hydroxyl group and a glycidyl group, after producing the polymer compound.

Examples of the crosslinkable functional group to be used include a functional group which produces a silanol group by hydrolysis, an epoxy group, a (meth)acrylic group and a glycidyl group. Since cross-linking treatment is easy, the functional group which produces the silanol group by hydrolysis, the epoxy group and the glycidyl group are preferable. Since the cross-linking treatment is performed at lower temperature, the functional group which produces the silanol group by hydrolysis is preferable.

The functional group which produces the silanol group by hydrolysis is a group which easily causes hydrolysis when it comes into contact with water, and produces the silanol group. Examples include a halogenated silyl group, an alkoxysilyl group, a phenoxysilyl group, and an acetoxysilyl group. The alkoxysilyl group, the phenoxysilyl group, and the acetoxysilyl group are preferable since no halogen is contained. Among the above, the alkoxysilyl group is most preferable since the silanol group is easily produced.

The ethylenically unsaturated polymerizable monomer having the functional group which produces the silanol group by hydrolysis is preferably an ethylenically unsaturated polymerizable monomer, in which a (meth)acrylic group and an alkoxysilyl group are bound via an alkyl chain having 1 to 20 carbon atoms or directly, represented by the following formula [2]:

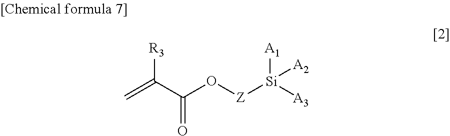

wherein R₃ represents a hydrogen atom or a methyl group; Z represents an alkyl group having 1 to 20 carbon atoms; and among A₁, A₂, and A₃, at least one is an alkoxy group capable of being hydrolyzed, and the others represent an alkyl group.

Examples of the ethylenically unsaturated polymerizable monomer having the alkoxysilyl group include (meth)acryloxyalkylsilane compounds such as 3-(meth)acryloxypropenyltrimethoxysilane, 3-(meth)acryloxypropylbis(trimethylsiloxy)methylsilane, 3-(meth)acryloxypropyldimethylmethoxysilane, 3-(meth)acryloxypropyldimethylethoxysilane, 3-(meth)acryloxypropylmethyldimethoxysilane, 3-(meth)acryloxypropylmethyldiethoxysilane, 3-(meth)acryloxypropyltrimethoxysilane, 3-(meth)acryloxypropyltriethoxysilane, 3-(meth)acryloxypropyltris(methoxyethoxy)silane, 8-(meth)acryloxyoctanyltrimethoxysilane, and 11-(meth)acryloxyundenyltrimethoxysilane. Among the above, 3-methacryloxypropyltrimethoxysilane, 3-methacryloxypropyltriethoxysilane, 3-methacryloxypropyldimethylmethoxysilane, and 3-methacryloxypropyldimethylethoxysilane are preferable from the viewpoint of excellent copolymerization capability with the ethylenically unsaturated polymerizable monomer having the alkylene glycol residue, and availability. The above ethylenically unsaturated polymerizable monomers having the alkoxysilyl group may be used alone or in combination of two or more kinds.

The composition ratio of the repeating unit (B) having the crosslinkable functional group in the polymer compound is preferably 1 to 20 mol %, more preferably 2 to 15 mol %, and still more preferably 2 to 10 mol %.

The compound used in the present invention may contain the functional group for fixing the biologically active substance. As an embodiment of the compound containing the functional group for fixing the biologically active substance, a polymer compound further containing a repeating unit (C) having the functional group for fixing the biologically active substance is preferable. It is preferable that the polymer compound containing the repeating unit (C) having the functional group for fixing the biologically active substance also contains the repeating unit (A) having the alkylene glycol residue or the phosphorylcholine group, and further contains the repeating unit (B) having the crosslinkable functional group. In the case that the polymer compound containing the repeating unit (C) having the functional group for fixing the biologically active substance contains the repeating unit (A) having the alkylene glycol residue or the phosphorylcholine group, the function of inhibiting nonspecific adsorption is more excellent, and thus, a background easily decreases, compared with the case of using the low molecular component for forming matrix in which the protein is less likely to be absorbed as disclosed in Patent Literature 2.

The repeating unit (C) having the functional group for fixing the biologically active substance is preferably one derived from an ethylenically unsaturated polymerizable monomer having the active ester group represented by the following formula [3]:

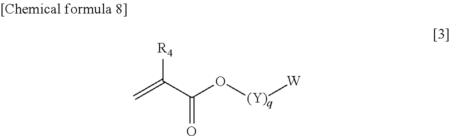

wherein R₄ represents a hydrogen atom or a methyl group; Y represents an alkylene group or alkylene glycol residue having 1 to 10 carbon atoms; W represents an active ester group; q represents an integer from 1 to 20; and provided that when q is 2 or more, the repeated Ys may be the same or different from each other.

Examples of the functional group for fixing the biologically active substance “W” include a p-nitrophenyl ester group, a N-hydroxysuccinimide ester group, a succinic acid imide ester group, a phthalic acid imide ester group, a 5-norbornene-2,3-dicarboxy imide ester group, an aldehyde group, an amino group, and an epoxy group. The p-nitrophenyl ester group or the N-hydroxysuccinimide ester group is preferable since the biologically active substance can be immobilized at lower pH level.

In the case of containing the repeating unit (C) having the functional group for fixing the biologically active substance, the composition ratio of the repeating unit (C) having the functional group for fixing the biologically active substance in the polymer compound is preferably 0.1 to 70 mol %, more preferably 1 to 20 mol %, and still more preferably 2 to 10 mol %.

The above polymer compound suitably used in the present invention may further contain other repeating units. For example, an ethylenically unsaturated polymerizable monomer having an alkyl group may be copolymerized. As the ethylenically unsaturated polymerizable monomer having the alkyl group, C1 to C15 alkyl ester of methacrylic acid is preferably used. The examples include methyl(meth)acrylate, ethyl(meth)acrylate, n-propyl(meth)acrylate, butyl(meth)acrylate, pentyl(meth)acrylate, hexyl(meth)acrylate, t-butyl(meth)acrylate, isopropyl(meth)acrylate, 2-ethylhexyl methacrylate, lauryl methacrylate, and cyclohexylmethacrylate. Among the above, n-butylmethacrylate, n-dodecylmethacrylate, or n-octylmethacrylate is preferable.

A synthesizing method of the polymer compound used in the present invention is not particularly limited. From the viewpoint of easiness in synthesis, it is preferable that a mixture at least containing the ethylenically unsaturated polymerizable monomer having the alkylene glycol residue, and, if necessary, further containing other ethylenically unsaturated polymerizable monomers is subjected to radical polymerization in a solvent in the presence of a polymerization initiator.

The solvent is not limited to any special solvent as long as each of ethylenically unsaturated polymerizable monomers can be dissolved therein. The examples include methanol, ethanol, t-butyl alcohol, benzene, toluene, tetrahydrofuran, dioxane, dichloromethane, and chloroform. These solvents are used alone or in combination of two or more kinds. In the case of applying the polymer compound on a plastic substrate, ethanol and methanol are preferable since the substrate is not denatured.

The polymerization initiator may be any of general radical initiator, and the examples include azo compounds such as 2,2′-azobisisobutylnitrile (hereinafter, it may be referred to as “AIBN”) and 1,1′-azobis(cyclohexane-1-carbonitrile), and organic peroxides such as benzoyl peroxide and lauryl peroxide.

In a chemical structure of the polymer compound used in the present invention, as long as it is a structure which is polymerized with the ethylenically unsaturated polymerizable monomer at least having the hydrophilic group inhibiting nonspecific adsorption, the bonding type thereof in the case that the polymer compound is a copolymer may be any type, such as a random, block or graft type.

A number average molecular weight of the polymer compound used in the present invention is preferably 5,000 or more, and more preferably 10,000 or more, since the polymer compound and an unreacted ethylenically unsaturated polymerizable monomer can be easily separated and refined.

The polymer compound was coated on a surface of the substrate, for example, by dissolving the compound in an organic solvent to prepare a solution having a concentration of 0.001 to 10 weight %, applying the obtained solution on the surface of the substrate by a known method such as dipping or spraying, and then, drying the resultant at room temperature or while heating. In the case of using the polymer compound having the crosslinkable functional group, after the above steps, the main chains of the polymer compound are crosslinked each other in any method suitable for the crosslinkable functional group. Upon coating a polymer compound in the case that the crosslinkable functional group has the functional group which produces the silanol group by hydrolysis, a mixed solution in which water is contained in an organic solvent may be used. Hydrolysis is caused by the contained water to produce the silanol group in the polymer compound. By additional heating, the main chains are bound each other, thus, the polymer compound becomes insoluble.

If the water content is low, the production of the silanol group is insufficient and a cross-linking bond becomes weak. On the other hand, if the water content is high, the polymer compound may be insoluble in the solvent. Theoretically, it is sufficient to contain water required for producing the silanol group by hydrolysis. From the view point of easiness in preparing the solution, the water content is preferably from about 0.01 to 15 weight %.

As the organic solvent, a single solvent such as ethanol, methanol, t-butyl alcohol, benzene, toluene, tetrahydrofuran, dioxane, dichloromethane, chloroform, acetone, or methyl ethyl ketone, or the mixed solvent thereof can be used. Among the above, ethanol or methanol is preferable since a plastic substrate is less likely to be denatured and can be easily dried. In addition, in the case of hydrolyzing the compound in the solution, ethanol or methanol is preferable since water is mixed therein in a desired ratio.

In the step of applying the solution prepared by dissolving the compound used in the present invention on the surface of the substrate followed by drying, in the case that the compound has a silanol group, a cross link is formed by dehydration condensation of the silanol group in the compound with a silanol group, a hydroxyl group, and/or an amino group in other compounds. Furthermore, similarly as in the above case, in the case that a hydroxyl group, a carbonyl group and/or an amino group are present on the surface of the substrate, the silanol group in the compound causes dehydration condensation and can chemically bond to the surface of the substrate. The covalent bond formed by the dehydration condensation of the silanol group is less likely to be hydrolized, therefore, the compound, with which the surface of the substrate is covered or attached, does not easily dissolve, and is not released from the substrate. The dehydration condensation of the silanol group can be facilitated by heating treatment. The heating treatment is preferably performed in the range of temperature that the compound is not denatured by heat, for example, from 60 to 120° C. for 5 minutes to 24 hours.

By applying the compound containing the hydrophilic group inhibiting the nonspecific adsorption on the surface of the substrate to cover or attach the surface of the substrate by the compound, a substrate for immobilizing the biologically active substance inhibiting nonspecific adsorption of the biologically active substance can be easily produced. Furthermore, in the case that the compound has a crosslinkable functional group, insolubility can be imparted to the compound on the substrate by crosslinking the compound. Thereby, the immobilizing substrate on which the compound is applied can be suitably used for a plate for ELISA and a substrate for a protein chip.

Various kinds of biologically active substances can be immobilized using the immobilizing method of the present invention. Examples of the biologically active substance to be immobilized include a nucleic acid, an aptamer, a protein, an antibody, an antigen, a lectine, a glycoprotein, and a sugar chain.

As the phosphate buffer used in the present invention, one in which any kind of phosphate is dissolved at a high concentration of 0.1 M or more, preferably 5.0 M or less, more preferably 4.0 M or less, still more preferably 0.6 M or more and 2.4 M or less, and most preferably 0.8 M or more and 1.4 M or less, can be used. When the concentration is less than the lower limit, there is a risk that the biologically active substance cannot be sufficiently immobilized and no signal can be detected. On the other hand, when the concentration is more than the upper limit, there is a risk that the biologically active substance can be denatured, so that the biologically active substance does not cause a specific reaction and function.

The phosphate used in the present invention is not particularly limited, and the examples include aluminum phosphate, ammonium phosphate, potassium phosphate, sodium phosphate, indium phosphate, samarium phosphate, potassium hydrogen phosphate, dipotassium hydrogen phosphate, calcium hydrogen phosphate, sodium hydrogen phosphate, disodium hydrogen phosphate, ammonium hydrogen phosphate, barium hydrogen phosphate, diammonium phosphate, dipotassium phosphate, 2-aminoethyl dihydrogen phosphate, ammonium dihydrogen phosphate, potassium dihydrogen phosphate, calcium dihydrogen phosphate, sodium dihydrogen phosphate, manganese dihydrogen phosphate, lithium dihydrogen phosphate, dibarium phosphate, hydroxyammonium phosphate, urea phosphate, lithium phosphate, diphenyl phosphate, triethyl phosphate, trioctyl phosphate, triphenyl phosphate, tributyl phosphate, trimethyl phosphate, boron phosphate and magnesium phosphate. Particularly preferred are dipotassium hydrogen phosphate and disodium hydrogen phosphate.

By bringing the solution prepared by dissolving the biologically active substance in the phosphate buffer having high concentration into contact with the immobilizing substrate, the biologically active substance can be easily immobilized.

A method for bringing the solution prepared by dissolving the biologically active substance into contact with the surface of the immobilizing substrate varies depending on the form of the substrate. For example, in the case of a 96-well plate, a solution only has to be poured in each well. In the case of a plate in a slide form, it is preferable that the solution is brought into contact with the surface by spot application.

### EXAMPLES

### Synthesis Example 1 of Polymer Compound

Polyethylene glycol methyl ether methacrylate (also known as methoxy polyethylene glycol methacrylate) (PEGMA; manufactured by Aldrich; average Mn=about 1,100) and 3-methacryloxypropyldimethylethoxysilane (MPDES; manufactured by Gelest, inc.) were respectively sequentially dissolved in dehydrated ethanol so as to set the concentration of each compound to 0.95 mol/L and 0.05 mol/L, so that a monomer mixed solution was prepared. Thereto, 2,2-azobisisobutyronitrile (AIBN; manufactured by Wako Pure Chemical Industries, Ltd.) was further added so as to set the concentration of the compound to 0.002 mol/L. The solution was stirred until the solution turned into a homogeneous state. Thereafter, the reactive components were reacted at 60° C. for 4 hours under an argon gas atmosphere, and then, the reaction solution was dropped into diethyl ether to collect a precipitate. The obtained polymer compound was measured by ¹H-NMR in a deuterated chloroform solvent, and the composition ratio of the polymer compound was calculated from a peak attributed to a methyl group which is bound to Si of MPDES appeared around 0.13 ppm, a peak attributed to an end methoxy group of PEGMA appeared around 3.4 ppm, and integral values thereof. The results are shown in Table 1.

### Synthesis Example 2 of Polymer Compound

Polyethylene glycol methyl ether methacrylate (also known as methoxy polyethylene glycol methacrylate) (PEGMA; manufactured by Aldrich; Average Mn=about 475) and 3-methacryloxypropyldimethylethoxysilane (MPDES; manufactured by Gelest, Inc.) were respectively sequentially dissolved in dehydrated ethanol so as to set the concentration of each compound to 0.95 mol/L and 0.05 mol/L, so that a monomer mixed solution was prepared. Thereto, 2,2-azobisisobutyronitrile (AIBN; manufactured by Wako Pure Chemical Industries, Ltd.) was further added so as to set the concentration of the compound to 0.002 mol/L. The solution was stirred until the solution turned into a homogeneous state. Thereafter, the reactive components were reacted at 60° C. for 1.5 hours under an argon gas atmosphere, and then, the reaction solution was dropped into diethyl ether to collect a precipitate. The obtained polymer compound was measured by ¹H-NMR in a deuterated ethanol solvent, and the composition ratio of the polymer compound was calculated from a peak attributed to a methyl group which is bound to Si of MPDES appeared around 0.15 ppm, a peak attributed to an end methoxy group of PEGMA appeared around 3.35 ppm, and integral values thereof. The results are shown in Table 1.

TABLE 1

| | | Synthesis | Synthesis |
|---|---|---|---|
| | | example 1 | example 2 |
| Charged | PEGMA (1100) | 90 | 0 |
| composition | PEGMA (475) | 0 | 90 |
| ratio | MPDES | 5 | 5 |
| Composition ratio | PEGMA (1100) | 94 | 0 |
| calculated from | PEGMA (475) | 0 | 96 |
| 1H-NMR | MPDES | 6 | 4 |

Unit: mol %

### Synthesis of p-Nitrophenyloxycarbonyl-Polyethylene Glycol Methacrylate (MEONP)

Polyethylene glycol monomethacrylate of 0.01 mol (product name: Blenmer PE-200 (n=4); manufactured by NOF Corp.) was dissolved in 20 mL of chloroform, and then, the solution was cooled to −30° C. Into this solution, a homogeneous solution prepared in advance and made of 0.01 mol of p-nitrophenyl chloroformate (manufactured by Sigma-Aldrich Corp.), 0.01 mol of triethylamine (manufactured by Wako Pure Chemical Industries, Ltd.) and 20 mL of chloroform was slowly dropped while the temperature was maintained at −30° C. After the reactive components were reacted at −30° C. for 1 hour, the solution was further stirred at room temperature for 2 hours. Thereafter, salts were filtrated off from the reaction solution, and the solvent was removed to obtain p-nitrophenyloxycarbonyl-polyethylene glycol methacrylate (MEONP). The resultant monomer was measured by ¹H-NMR in deuterated chloroform solvent. As a result, it was confirmed that 4.5 units of ethylene glycol residues were contained.

### Synthesis Example 3 of Polymer Compound

2-methacryloyloxyethyl phosphorylcholine (MPC), butylmethacrylate (BMA), and MEONP were respectively sequentially dissolved in dehydrated ethanol so as to set the concentration of each compound to 0.25 mol/L, 0.70 mol/L, and 0.05 mol/L, so that a monomer mixed solution was prepared. Thereto, AIBN was added so as to set the concentration of the compound to 0.002 mol/L. The solution was stirred until the solution turned into a homogeneous state. Thereafter, the reactive components were reacted at 60° C. for 3 hours under an argon gas atmosphere, and then, the reaction solution was dropped into a mixed solvent of diethyl ether and chloroform to collect a precipitate. The obtained polymer compound was measured by ¹H-NMR, and the composition ratio of the polymer compound was calculated from peaks attributed to methylene of BMA appeared around 1.46 ppm and 1.65 ppm, a peak attributed to trimethyl of MPC appeared around 3.34 ppm, peaks attributed to a benzene ring of MEONP appeared around 7.6 ppm and 8.4 ppm, and integral values thereof. The results are shown in Table 2.

### Synthesis Example 4 of Polymer Compound

Polyethylene glycol methyl ether methacrylate (also known as methoxy polyethylene glycol methacrylate) (PEGMA; manufactured by Aldrich; Average Mn=about 1,100), p-nitrophenyloxycarbonyl-polyethylene glycol methacrylate (MEONP), and 3-methacryloxypropyldimethylethoxysilane (MPDES; manufactured by Gelest, Inc.) were respectively sequentially dissolved in dehydrated ethanol so as to set the concentration of each compound to 0.45 mol/L, 0.025 mol/L, and 0.025 mol/L, so that a monomer mixed solution was prepared. Thereto, 2,2-azobisisobutyronitrile (AIBN; manufactured by Wako Pure Chemical Industries, Ltd.) was further added so as to set the concentration of the compound to 0.002 mol/L. The solution was stirred until the solution turned into a homogeneous state. Thereafter, the reactive components were reacted at 60° C. for 1 hour under an argon gas atmosphere, and then, the reaction solution was dropped into diethyl ether to collect a precipitate. The obtained polymer compound was measured by ¹H-NMR in a deuterated ethanol solvent, and the composition ratio of the polymer compound was calculated from a peak attributed to a methyl group which is bound to Si of MPDES appeared around 0.16 ppm, a peak attributed to an end methoxy group of PEGMA appeared around 3.35 ppm, peaks attributed to a benzene ring of MEONP appeared around 7.6 ppm and 8.4 ppm, and integral values thereof. The results are shown in Table 2.

TABLE 2

| | Composition ratio calculated from 1H-NMR | | |
|---|---|---|---|
| | MPC | BMA | MEONP |
| Synthesis example 3 | 21 | 76 | 3 |
| | PEGMA | MPDES | MEONP |
| Synthesis example 4 | 92 | 5 | 3 |

Unit: mol %

### (Production of Immobilizing Substrate)

A substrate was produced by molding a saturated cyclic polyolefin resin in the form of a 96-well plate (well dimension: diameter of bottom surface 6.4 mm×height 11 mm). Oxidation treatment was performed on a surface of the substrate by plasma treatment in an oxygen atmosphere. Thus obtained substrate was dipped in a 0.3 weight % ethanol solution of the polymer compound obtained in Synthesis examples 1 to 4, followed by heating at 65° C. for 4 hours. Thereby, a layer containing the polymer compound obtained in Synthesis examples 1 to 4 was introduced onto the surface of the substrate. The immobilizing substrate produced using the polymer compound of Synthesis example 1 is referred to as an immobilizing substrate 1, the immobilizing substrate produced using the polymer compound of Synthesis example 2 is referred to as an immobilizing substrate 2, the immobilizing substrate produced using the polymer compound of Synthesis example 3 is referred to as an immobilizing substrate 3, and the immobilizing substrate produced using the polymer compound of Synthesis example 4 is referred to as an immobilizing substrate 4.

### Experiment 1

The relationship between immobilization of a primary antibody and a concentration of phosphate was studied.

### Example 1

### Preparation of Immobilizing Solution

A solution was prepared in which a primary antibody, anti-mouse IgG2a, was contained at a concentration of 1.2 μg/mL in an aqueous solution of 1.2 M dipotassium hydrogen phosphate (product number: 164-04295; manufactured by Wako Pure Chemical Industries, Ltd.).

### (Immobilizing Treatment)

### Step 1

The prepared immobilizing solution was poured in each well on an immobilizing substrate 1 at a rate of 100 μl/well, and left at room temperature for 4 hours. After an immobilizing reaction, the immobilizing substrate was washed by 1×SSC buffer (diluted SSC 20× Buffer (product name; manufactured by Zymed Laboratories, Inc.)), to which a 0.05 wt % nonionic surfactant (product name: Tween 20; manufactured by Roche Diagnostics K.K.) was added, at room temperature for 5 minutes.

### Step 2

Thereafter, adsorption inhibiting treatment was not performed.

### Step 3 (Antigen-Antibody Reaction 1)

An FBS (fetal bovine serum) solution was prepared by diluting FBS (fetal bovine serum) to 10% with a PBS buffer (a buffer dissolving 9.6 g/L of tissue-culturing Dulbecco's PBS(−) (product name; manufactured by Nissui Pharmaceutical Co., Ltd.)). To this solution, a mouse IgG2a being an antigen was added to produce a 20 nmol/L solution. This solution was diluted with the FBS (fetal bovine serum) solution diluted to 10% with a PBS buffer (a buffer dissolving 9.6 g/L of tissue-culturing Dulbecco's PBS(−) (product name; manufactured by Nissui Pharmaceutical Co., Ltd.)) one time, 2 times, 3 times and 4 times, so as to yield diluted solutions. These diluted solutions and the 10% FBS solution in which no mouse IgG2a was contained as the antigen were respectively brought into contact with an immobilizing substrate at 37° C. for 2 hours to develop an antigen-antibody reaction. After the antigen-antibody reaction, the immobilizing substrate was washed by the 1×SSC buffer (diluted SSC 20× Buffer (product name; manufactured by Zymed Laboratories, Inc.)), to which a 0.05 wt % nonionic surfactant (product name: Tween 20; manufactured by Roche Diagnostics K.K.) was added, at room temperature for 5 minutes.

### Step 4 (Antigen-Antibody Reaction 2)

After the washing, a HRP-labeled anti-mouse IgG2a as a second antibody was added to a PBS buffer (a buffer dissolving 9.6 g/L of tissue-culturing Dulbecco's PBS(−) (product name; manufactured by Nissui Pharmaceutical Co., Ltd.)) to prepare a 20 nmol/L solution. An antigen-antibody reaction between this solution and the immobilizing substrate was developed at 37° C. for 2 hours. After the antigen-antibody reaction, the immobilizing substrate was washed by the 1×SSC buffer (diluted SSC 20× Buffer (product name; manufactured by Zymed Laboratories, Inc.)), to which a 0.05 wt % nonionic surfactant (product name: Tween 20; manufactured by Roche Diagnostics K.K.) was added, at room temperature for 5 minutes.

### Step 5 (Coloring)

Finally, a coloring reaction was carried out with a TMBZ coloring kit (product name; manufactured by Sumitomo Bakelite Co., Ltd.), which is a HRP coloring reagent.

The light absorption amount at 450 nm of the substrate, in which coloring reaction was carried out, was measured. A plate reader (manufactured by TECAN) was used for measuring the light absorption amount. The results of signal intensity are shown in Table 3.

### Comparative Example 1

### Preparation of Immobilizing Solution

A solution was prepared in which a primary antibody, anti-mouse IgG2a, was contained at a concentration of 1.2 μg/mL in an aqueous solution of 0.05 M dipotassium hydrogen phosphate (product number: 164-04295; manufactured by Wako Pure Chemical Industries, Ltd.).

Hereafter, similar steps as in Example 1 were carried out. The results are shown in Table 3.

By comparing Example 1 and Comparative example 1, it was confirmed that the signal corresponding to the amount of the antigen was detected and the primary antibody was immobilized in Example 1 using the phosphate buffer having high concentration.

TABLE 3
Values of signal intensity

| | Diluting magnification | | | | |
|---|---|---|---|---|---|
| | 1 | 2 | 3 | 4 | no antigen |
| Example 1 | 1.33 | 1.05 | 0.65 | 0.37 | 0.09 |
| Comparative | 0.09 | 0.08 | 0.06 | 0.09 | 0.08 |
| example 1 | | | | | |

### Experiment 2

An immobilizing substrate 2 and a non-coated substrate were compared.

### Example 2

As an immobilizing substrate, an immobilizing substrate 2 was used. The same experiment process as in Example 1 was carried out. The results are shown in Table 4.

### Comparative Example 2

As a substrate, a substrate after plasma treatment in an oxygen atmosphere was used without applying the polymer compound. The same experiment process as in Example 1 was carried out. The results are shown in Table 4.

By comparing Example 2 and Comparative example 2, it was confirmed that the primary antibody was able to be immobilized even to a surface having no alkylene glycol residue using the phosphate buffer having a high concentration, however, in Comparative example 2 using a substrate having no alkylene glycol residue, a background was high and the range of detecting an antigen concentration and the signal was narrow. It was also confirmed that, in Example 2 using a substrate having the alkylene glycol residue, a background did not increase and a wide range of detection was shown.

TABLE 4
Values of signal intensity

| | Diluting magnification | | | | |
|---|---|---|---|---|---|
| | 1 | 2 | 3 | 4 | no antigen |
| Example 2 | 1.42 | 1.11 | 0.69 | 0.35 | 0.08 |
| Comparative | 1.43 | 1.30 | 1.38 | 1.29 | 1.02 |
| example 2 | | | | | |

### Experiment 3

Using an immobilizing substrate 3 and an immobilizing substrate 4, the relationship between immobilization of a primary antibody and a concentration of phosphate was studied.

### Examples 3 and 4

### Preparation of Immobilizing Solution

A solution was prepared in which a primary antibody, anti-mouse IgG2a, was contained at a concentration of 1.2 μg/mL in an aqueous solution of 1.2 M dipotassium hydrogen phosphate (product number: 164-04295; manufactured by Wako Pure Chemical Industries, Ltd.).

### (Immobilizing Treatment)

The same Step 1, Step 3, Step 4 and Step 5 as in Example 1 were carried out except that the following Step 2 was carried out in place of Step 2 in Example 1. The results of signal intensity are shown in Table 5.

### Step 2

After Step 1, the substrate was dipped in 0.1 mol/L ethanolamine (manufactured by Wako Pure Chemical Industries, Ltd.; Cica special grade) and an aqueous solution (pH9.5) of 0.1 mol/L tris buffer (manufactured by Sigma) for 1 hour, thus, remaining MEONP parts were deactivated.

### Comparative examples 3 and 4

### Preparation of Immobilizing Solution

A solution was prepared in which a primary antibody, anti-mouse IgG2a, was contained at a concentration of 1.2 μg/mL in an aqueous solution (pH 8.5) of 1.2 M boric-acid (product number: 027-02191; manufactured by Wako Pure Chemical Industries, Ltd.).

Hereafter, similar steps as in Examples 3 and 4 were carried out. The results are shown in Table 5.

### Comparative examples 5 and 6

### Preparation of Immobilizing Solution

The same process as in Examples 3 and 4 was carried out except that a 0.05 M dipotassium hydrogen phosphate aqueous solution was used in place of the aqueous solution of 1.2 M dipotassium hydrogen phosphate (product number: 164-04295; manufactured by Wako Pure Chemical Industries, Ltd.). The results are shown in Table 5.

By comparing Examples 3 and 4, and Comparative examples 3 and 4, the signal corresponding to the amount of the antigen was detected in Examples 3 and 4 using the phosphate solution. Therefore, it was conformed that the primary antibody was immobilized. In addition, by comparing Examples 3 and 4, and Comparative examples 5 and 6, it was confirmed that higher signal was obtained using phosphate having higher concentration.

TABLE 5
Values of signal intensity

| | | Diluting magnification | | | | |
|---|---|---|---|---|---|---|
| | Immobilizing substrate | 1 | 2 | 3 | 4 | no antigen |
| Example 3 | Immobilizing substrate 3 | 1.33 | 1.05 | 0.65 | 0.37 | 0.09 |
| Example 4 | immobilizing substrate 4 | 1.54 | 1.14 | 0.78 | 0.52 | 0.09 |
| Comparative example 3 | Immobilizing substrate 3 | 0.09 | 0.08 | 0.06 | 0.09 | 0.08 |
| Comparative example 4 | Immobilizing substrate 4 | 0.09 | 0.11 | 0.08 | 0.09 | 0.10 |
| Comparative example 5 | Immobilizing substrate 3 | 0.15 | 0.12 | 0.11 | 0.12 | 0.09 |
| Comparative example 6 | Immobilizing substrate 4 | 0.11 | 0.12 | 0.09 | 0.09 | 0.09 |

1-4. (canceled)

5. The method of immobilizing the biologically active substance according to claim 20, wherein the repeating unit (A) having the alkylene glycol residue is derived from an ethylenically unsaturated polymerizable monomer having an alkylene glycol residue represented by the following formula [1′]: 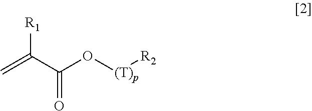
wherein R₁ represents a hydrogen atom or a methyl group; R₂ represents a hydrogen atom or an alkyl group having 1 to 20 carbon atoms; T represents an alkylene glycol residue having 1 to 10 carbon atoms; p represents an integer from 1 to 100; and provided that when p is an integer of 2 or more and 100 or less, the repeated Ts may be the same or different from each other.

6. The method of immobilizing the biologically active substance according to claim 5, wherein the ethylenically unsaturated polymerizable monomer having the alkylene glycol residue is methoxy polyethylene glycol(meth)acrylate.

7. The method of immobilizing the biologically active substance according to claim 6, wherein an average chain number of ethylene glycol in the methoxy polyethylene glycol(meth)acrylate is in the range from 3 to 100.

8. The method of immobilizing the biologically active substance according to claim 20, wherein the polymer compound contains a repeating unit (B) having a crosslinkable functional group.

9. The method of immobilizing the biologically active substance according to claim 8, wherein the crosslinkable functional group of the repeating unit (B) is at least one functional group selected from alkoxysilyl, epoxy, and (meth)acrylic groups.

10. The method of immobilizing the biologically active substance according to claim 8, wherein the repeating unit (B) is an ethylenically unsaturated polymerizable monomer having alkoxysilyl represented by the following formula [2]: 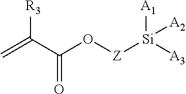
wherein R₃ represents a hydrogen atom or a methyl group; Z represents an alkyl group having 1 to 20 carbon atoms; and among A₁, A₂, and A₃, at least one is an alkoxy group capable of being hydrolyzed, and the others represent an alkyl group.

11-12. (canceled)

13. The method of immobilizing the biologically active substance according to claim 20, wherein the polymer compound contains a functional group for fixing the biologically active substance, the functional group for fixing the biologically active substance being a p-nitrophenyl ester group.

14. The method of immobilizing the biologically active substance according to claim 20, wherein the phosphate concentration of the phosphate buffer is 5M or less.

15. The method of immobilizing the biologically active substance according to claim 20, wherein the biologically active substance is a nucleic acid, an aptamer, a protein, an antibody, an antigen, a lectin, a glycoprotein, or a sugar chain.

16. The method of immobilizing the biologically active substance according to claim 20, wherein the substrate is made of plastic.

17. The method of immobilizing the biologically active substance according to claim 16, wherein the plastic is saturated cyclic polyolefin, polyolefin, or polystyrene.

18. The method of immobilizing the biologically active substance according to claim 20, wherein the substrate is made of glass.

19. The method of immobilizing the biologically active substance according to claim 20, wherein the substrate is in the form of a slide, a 96-well plate, a 384-well plate, a 1536-well plate, a microchannel, a bead, a tube, or a container.

20. A method of immobilizing a biologically active substance comprising the steps of:
providing an immobilizing substrate which comprises, on a surface of the substrate, a polymer compound containing a hydrophilic group inhibiting nonspecific adsorption;
providing an immobilizing solution prepared by dissolving the biologically active substance in a phosphate buffer having a phosphate concentration of 0.6 M or more; and
bringing the immobilizing solution into contact with the surface of the immobilizing substrate to immobilize the biologically active substance on the surface, wherein
said polymer compound contains a repeating unit (A) which has the hydrophilic group selected from a group consisting of an alkylene glycol residue and a phosphorylcholine group and is derived from an ethylenically unsaturated polymerizable monomer represented by the following formula [1]: 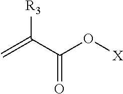
wherein R₁ represents a hydrogen atom or a methyl group; and X represents a group having the alkylene glycol residue or the phosphorylcholine group; and
said polymer compound contains a repeating unit (C) which is derived from an ethylenically unsaturated polymerizable monomer having an active ester group as the functional group for fixing a biologically active substance, said ethylenically unsaturated polymerizable monomer is represented by the following formula [4]: 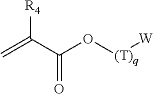
wherein R₄ represents a hydrogen atom or a methyl group; Y represents an alkylene group or alkylene glycol residue having 1 to 10 carbon atoms; W represents an active ester group; q represents an integer from 1 to 20; and provided that when q is 2 or more, the repeated Ys may be the same or different from each other.

21. The method of immobilizing a biologically active substance according to claim 20, wherein the polymer compound contains the repeating unit (C) at 0.1 to 70 mol % of the total amount of all repeating units.

22. The method of immobilizing the biologically active substance according to claim 5, wherein the polymer compound contains a repeating unit (B) having a crosslinkable functional group.

23. The method of immobilizing the biologically active substance according to claim 6, wherein the polymer compound contains a repeating unit (B) having a crosslinkable functional group.

24. The method of immobilizing the biologically active substance according to claim 7, wherein the polymer compound contains a repeating unit (B) having a crosslinkable functional group.

25. The method of immobilizing the biologically active substance according to claim 9, wherein the repeating unit (B) is an ethylenically unsaturated polymerizable monomer having alkoxysilyl represented by the following formula [2]: 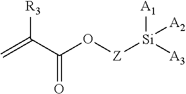
wherein R₃ represents a hydrogen atom or a methyl group; Z represents an alkyl group having 1 to 20 carbon atoms; and among A₁, A₂, and A₃, at least one is an alkoxy group capable of being hydrolyzed, and the others represent an alkyl group.
